(19) United States
(12) Patent Application Publication
(10) Pub. No.: US 2017/0172402 A1
(43) Pub. Date: Jun. 22, 2017

(54) ENDOSCOPE

(71) Applicant: OLYMPUS CORPORATION, Tokyo (JP)

(72) Inventor: Katsuhiro WAKABAYASHI, Tokyo (JP)

(73) Assignee: OLYMPUS CORPORATION, Tokyo (JP)

(21) Appl. No.: 15/447,786

(22) Filed: Mar. 2, 2017

Related U.S. Application Data
(63) Continuation of application No. PCT/JP2015/057195, filed on Mar. 11, 2015.

(30) Foreign Application Priority Data
Sep. 5, 2014 (JP) 2014-181417

(51) Int. Cl. A61B 1/12; A61B 8/00; A61B 8/12; A61B 8/14; A61B 1/018; A61B 1/05

(52) U.S. Cl. CPC A61B 1/128; A61B 1/018; A61B 1/05; A61B 8/12; A61B 8/14; A61B 8/4483

(57) ABSTRACT

An endoscope includes an insertion section configured to be inserted into a subject, a distal end portion disposed at a distal end of the insertion section, the distal end portion including an observing section configured to observe the subject, a holding section configured to hold the observing section, a heat medium that changes in phase from liquid to gas when exceeding a predetermined temperature, and a heat receiving chamber configured to house the heat medium, and a heat radiation chamber communicating with the heat receiving chamber, the heat medium gasified in the heat receiving chamber being capable of entering the heat radiation chamber.

### CROSS REFERENCE TO RELATED APPLICATION

This application is a continuation application of PCT/JP2015/057195 filed on Mar. 11, 2015 and claims benefit of Japanese Application No. 2014-181417 filed in Japan on Sep. 5, 2014, the entire contents of which are incorporated herein by this reference.

### BACKGROUND OF THE INVENTION

1. Field of the Invention

The present invention relates to an endoscope including, in an insertion section, an observing section that generates heat.

2. Description of the Related Art

An endoscope used in a medical field includes an endoscope having a form including, in a distal end portion of an insertion section insertable into a subject, a heat generating section configured to generate heat according to operation. The heat generating section included in the distal end portion of the insertion section in the endoscope includes, for example, an ultrasound transducer configured to transmit and receive ultrasound, a light source apparatus configured to emit illumination light, or an image pickup apparatus configured to pick up an optical image.

For example, Japanese Patent Application Laid-Open Publication No. 2008-43440 discloses a technique for providing, in an insertion section of an endoscope, a channel in which a coolant flows and cooling a heat generating section, the coolant being liquid or gas.

### SUMMARY OF THE INVENTION

An endoscope according to an aspect of the present invention includes: an insertion section configured to be inserted into a subject; a distal end portion disposed at a distal end of the insertion section, the distal end portion including an observing section configured to observe the subject, a holding section configured to hold the observing section, a heat medium that changes in phase from liquid to gas when exceeding a predetermined temperature, and a heat receiving chamber configured to house the heat medium; and a heat radiation chamber communicating with the heat receiving chamber, the heat medium gasified in the heat receiving chamber being capable of entering the heat radiation chamber.

An endoscope according to another aspect of the present invention includes: an ultrasound transmitting/receiving section; and a holding section configured to form a heat receiving chamber, which is a space in which a heat medium that changes in phase from liquid to gas when exceeding a predetermined temperature is encapsulated, between the holding section and the ultrasound transmitting/receiving section and hold the ultrasound transmitting/receiving section in the heat medium such that at least a part of an outer surface of the ultrasound transmitting/receiving section is exposed to the heat receiving chamber. A plurality of slits are formed in a portion in contact with the heat medium in the ultrasound transmitting/receiving section.

### BRIEF DESCRIPTION OF THE DRAWINGS

FIG. 1 is a diagram for explaining a configuration of an endoscope in a first embodiment;

FIG. 2 is a diagram showing an exterior of a distal end portion of an insertion section in the first embodiment;

FIG. 3 is a sectional view of FIG. 2;

FIG. 4 is a IV-IV sectional view of FIG. 3;

FIG. 5 is a V-V sectional view of FIG. 3;

FIG. 6 is a diagram showing a modification of the distal end portion of the insertion section of the endoscope in the first embodiment;

FIG. 7 is a sectional view of a distal end portion of an insertion section of an endoscope in a second embodiment;

FIG. 8 is a VIII-VIII sectional view of FIG. 7;

FIG. 9 is a sectional view of a distal end portion of an insertion section of an endoscope in a third embodiment;

FIG. 10 is a sectional view of a distal end portion of an insertion section of an endoscope in a fourth embodiment; and

FIG. 11 is a diagram showing a cross section of a distal end portion of an insertion section and a heat radiation chamber provided in an operation section of an endoscope in a fifth embodiment.

### DETAILED DESCRIPTION OF THE PREFERRED EMBODIMENT(S)

Preferred modes of the present invention are explained below with reference to the drawings. Note that, in respective figures used in the following explanation, scales are differentiated for each of components to show the respective components in sizes recognizable on the drawings. The present invention is not limited only to numbers of the components, shapes of the components, ratios of sizes of the components, and relative positional relations among the respective components described in the figures.

### First Embodiment

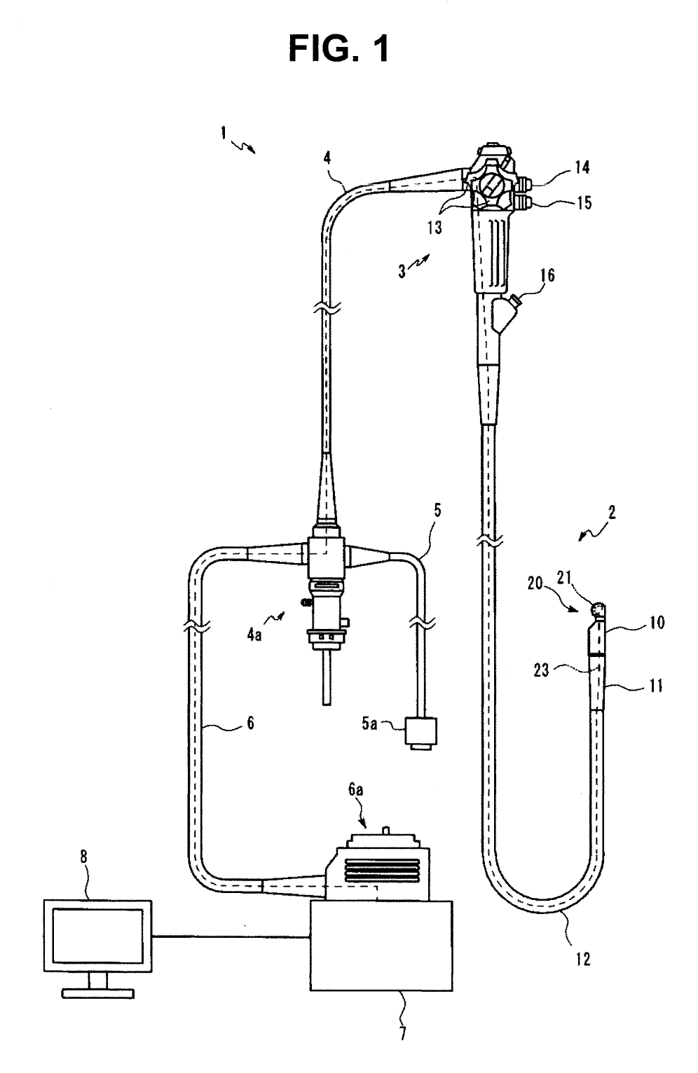

An endoscope 1 in the present embodiment shown in FIG. 1 has a form called ultrasound endoscope including, in a distal end portion 10 of an insertion section 2 inserted into an inside of a subject such as a human body, as an example, an ultrasound transmitting/receiving section 21 configured to transmit and receive ultrasound. Note that the subject, into which the insertion section 2 of the endoscope 1 is inserted, is not limited to an organism such as the human body and may be an inanimate matter such as a machine or a building.

Detailed explanation of an overall configuration of the endoscope 1 is omitted because the overall configuration of the endoscope 1 is well known. However, a schematic configuration of the endoscope 1 is explained below. The endoscope 1 mainly includes the insertion section 2 insertable into a body of the subject, an operation section 3 located at a proximal end of the insertion section 2, and a universal cord 4 extending from a side portion of the operation section 3.

The insertion section 2 is configured by concatenating the distal end portion 10 disposed at a distal end, a bendable bending section 11 disposed on a proximal end side of the distal end portion 10, and a flexible tube section 12 having flexibility disposed on the proximal end side of the bending section 11 and connected to a distal end side of the operation section 3. Note that the endoscope 1 may be an endoscope having a form called rigid endoscope not including a part having flexibility in the insertion section 2.

An observing section 20 configured to observe the subject is disposed at the distal end portion 10 of the insertion section 2. The observing section 20 includes at least one of a component configured to optically observe the subject and a component configured to acoustically observe the subject. For example, when the observing section 20 includes the component configured to optically observe the subject, the observing section 20 includes an image pickup apparatus and an illuminating apparatus. For example, when the observing section 20 includes the component configured to acoustically observe the subject, the observing section 20 includes the ultrasound transmitting/receiving section 21.

In the present embodiment, as an example, the observing section 20 includes the image pickup apparatus and the illuminating apparatus (not shown in the figure) and the ultrasound transmitting/receiving section 21. The ultrasound transmitting/receiving section 21 includes a plurality of ultrasound transducers 22 as explained below and is a heat generating section configured to generate heat according to operation.

Note that, when the image pickup apparatus includes an electronic circuit including an image pickup device, the image pickup apparatus included in the observing section 20 is also the heat generating section. When the illuminating apparatus includes a light source apparatus such as a light emitting diode, the illuminating apparatus included in the observing section 20 is also the heat generating section.

At the distal end portion 10, a treatment instrument projecting port 17a, which is an opening portion communicating with a treatment instrument channel 17 (not shown in FIG. 1), and the like are provided. At the distal end portion 10, an air/water feeding port for delivering gas and liquid is provided.

In the operation section 3, an angle operation knob 13 for operating bending of the bending section 11 is provided. In the operation section 3, a suction switch 14 configured to perform control of suction action from the treatment instrument projecting port provided at the distal end portion 10 and an air/water feeding switch 15 configured to perform control of air feeding action and water feeding action from the air/water feeding port provided at the distal end portion 10 are disposed. In the operation section 3, a treatment instrument inserting port 16, which is an opening portion communicating with the treatment instrument channel 17, is disposed.

An endoscope connector 4a connected to the not-shown light source apparatus is provided at a proximal end portion of the universal cord 4. Light emitted from the light source apparatus is transmitted through an optical fiber cable inserted through the universal cord 4, the operation section 3, and the insertion section 2 and emitted from the illuminating apparatus at the distal end portion 10. Note that, when the illuminating apparatus disposed at the distal end portion 10 includes the light source apparatus, the connection to the light source apparatus is unnecessary.

A video cable 5 and an ultrasound cable 6 extend from the endoscope connector 4a. The video cable 5 is detachably connected to a not-shown camera control unit via an electric connector 5a. The camera control unit is an apparatus configured to output, to an image display apparatus 8, an image picked up by the image pickup apparatus provided at the distal end portion 10.

The ultrasound cable 6 is detachably connected to an ultrasound observation control apparatus 7 via an ultrasound connector 6a. The ultrasound connector 6a is electrically connected to the ultrasound transmitting/receiving section 21 included in the observing section 20 via a coaxial cable bundle 23 inserted through the ultrasound cable 6, the universal cord 4, the operation section 3, and the insertion section 2.

The ultrasound observation control apparatus 7 is an apparatus configured to perform control of transmitting/receiving action of ultrasound by the ultrasound transmitting/receiving section 21 and generation of an ultrasonogram and output the ultrasonogram to the image display apparatus 8.

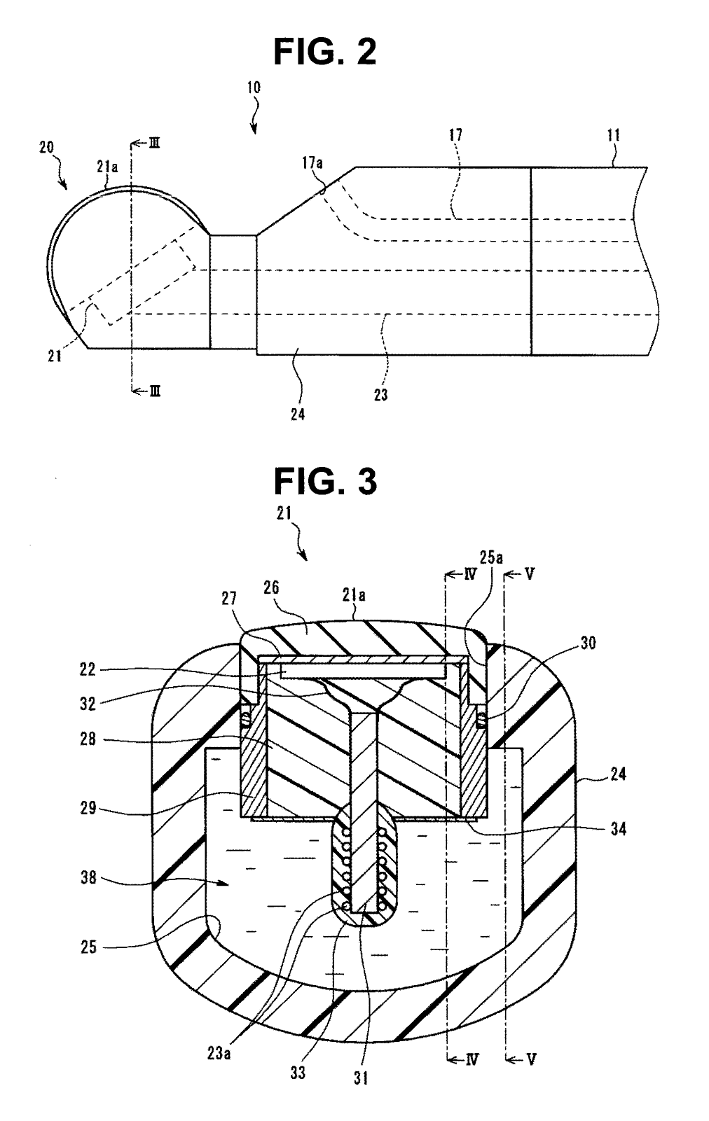

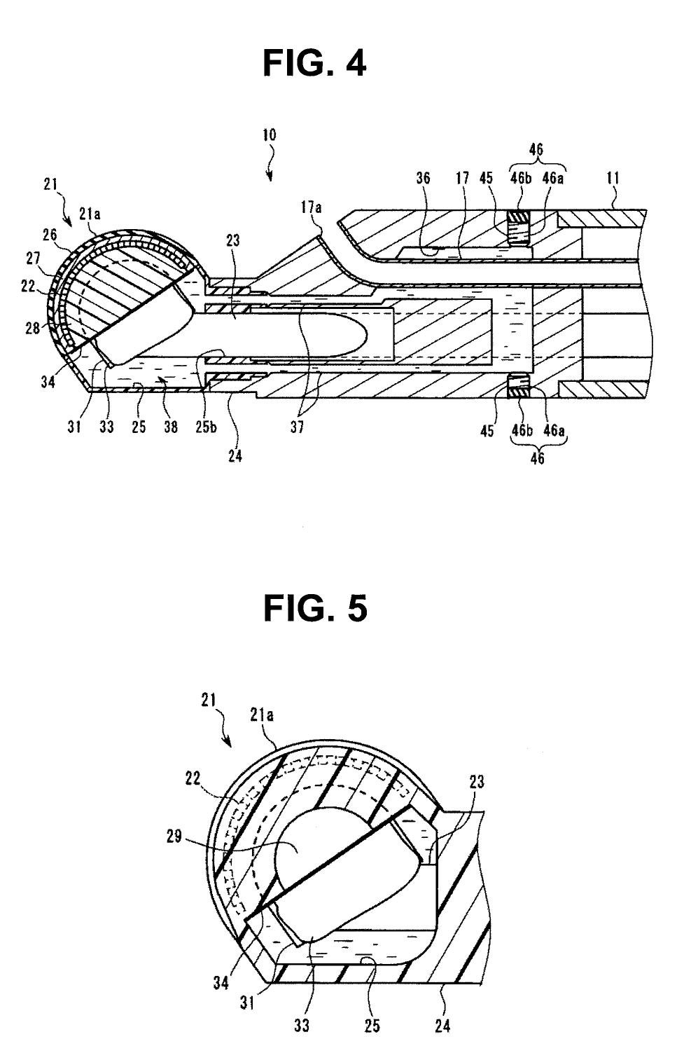

A configuration of the distal end portion 10 of the insertion section 2 is explained. FIG. 2 is a diagram showing an exterior of the distal end portion 10. FIG. 3 is a sectional view of FIG. 2. FIG. 4 is a IV-IV sectional view of FIG. 3. FIG. 5 is a V-V sectional view of FIG. 3.

The distal end portion 10 includes a holding section 24 fixed to a distal end of the bending section 11. The holding section 24 is made of a rigid material formed of metal, synthetic resin, or the like. The holding section 24 holds a distal end portion of the treatment instrument channel 17, the observing section 20, and the like, which are components included in the distal end portion 10.

As explained above, in the present embodiment, as an example, the observing section 20, which is the heat generating section, is the ultrasound transmitting/receiving section 21. The ultrasound transmitting/receiving section 21 is fixed to the holding section 24 in a state in which a surface 21a for transmitting and receiving ultrasound is exposed to an outside of the holding section 24.

Note that the holding section 24 may be configured by a plurality of members. For example, a form may be adopted in which a part fixed to the bending section 11 and a part for holding the ultrasound transmitting/receiving section 21 of the holding section 24 are configured from members made of different materials.

As shown in FIG. 4, the ultrasound transmitting/receiving section 21 includes a plurality of ultrasound transducers 22. The ultrasound transducer 22 is not particularly limited as long as the ultrasound transducer 22 is capable of converting an electric signal and ultrasound into each other. However, for example, a piezoelectric device or an electrostriction device such as piezoelectric ceramics or an ultrasound transducer (MUT: micromachined ultrasonic transducer) by a micromachine technique can be applied. In the present embodiment, as an example, the ultrasound transducer 22 is the piezoelectric device.

The number and a form of an array of the plurality of ultrasound transducers 22 in the ultrasound transmitting/receiving section 21 are not particularly limited. The ultrasound transmitting/receiving section 21 may have a form including a one-dimensional array (1D array) configured by arraying the plurality of ultrasound transducers 22 in a row or a form including a two-dimensional array (2D array) configured by arraying the plurality of ultrasound transducers 22 in a matrix shape. The ultrasound transmitting/receiving section 21 may have a form that is generally called 1.25D array and in which width of an ultrasound beam is variable and a form that is generally called 1.5D array and in which width and a focal length of an ultrasound beam are variable by arraying the plurality of ultrasound transducers 22 in a matrix shape.

The ultrasound transmitting/receiving section 21 in the present embodiment has, as an example, a form called convex type. The plurality of ultrasound transducers 22 are arrayed in a row along a circumferential direction of a cylindrical surface. Note that the ultrasound transmitting/receiving section 21 may be, for example, a linear type or a radial type.

As shown in FIG. 3 and FIG. 4, in the holding section 24, a heat receiving chamber 25, which is a hollow section, is provided on the inside. In the holding section 24, an opening section 25a configured to cause the heat receiving chamber 25 and an external space of the holding section 24 to communicate with each other is provided.

The ultrasound transmitting/receiving section 21 is disposed in the opening section 25a with the surface 21a for transmitting and receiving ultrasound directed toward the outside of the holding section 24. The opening section 25a is sealed by the ultrasound transmitting/receiving section 21 disposed in the opening section 25a. For example, as shown in FIG. 3, a seal member 30 for improving water tightness between an outer circumferential surface of the ultrasound transmitting/receiving section 21 and an inner circumferential surface of the opening section 25a is sandwiched between the outer circumferential surface and the inner circumferential surface. Note that not-shown resin such as an adhesive is also used for the sealing of the opening section 25a.

In the holding section 24, a cable insert-through port 25b configured to cause the heat receiving chamber 25 and an internal space of the insertion section 2 to communicate with each other is provided. The coaxial cable bundle 23 is inserted through the cable insert-through port 25b. That is, a distal end portion of the coaxial cable bundle 23 is located in the heat receiving chamber 25. The cable insert-through port 25b is sealed by not-shown resin in a state in which the coaxial cable bundle 23 is inserted through the cable insert-through port 25b.

The ultrasound transmitting/receiving section 21 includes an acoustic lens 26, an acoustic matching layer 27, a backing material 28, and an electric circuit board 31 besides the ultrasound transducer 22.

As shown in FIG. 3, the acoustic lens 26 is provided to be exposed to the surface 21a for transmitting and receiving ultrasound of the ultrasound transmitting/receiving section 21. That is, the acoustic lens 26 is a part exposed to the outside of the holding section 24 of the ultrasound transmitting/receiving section 21. The acoustic matching layer 27 is interposed between the ultrasound transducer 22 and the acoustic lens 26. The acoustic matching layer 27 performs acoustic impedance matching between the ultrasound transducer 22 and the acoustic lens 26.

The backing material 28 is disposed on an opposite side of the acoustic matching layer 27 of the ultrasound transducer 22, that is, on a side serving as an inner side of the holding section 24. The backing material 28 is made of electrically insulative resin or the like. The backing material 28 attenuates ultrasound radiated from the ultrasound transducer 22 toward the inner side of the holding section 24 and ultrasound traveling from the inner side of the holding section 24 toward the ultrasound transducer 22.

In the present embodiment, as an example, an outer circumferential member 29, which is a tabular member surrounding side surfaces of the ultrasound transducer 22, is disposed on a side surface, which is a surface opposed to the inner circumferential surface of the opening section 25a, of the ultrasound transmitting/receiving section 21. The seal member 30 is sandwiched between the outer circumferential member 29 and the inner circumferential surface of the opening section 25a.

The backing material 28 is filled in a space surrounded by the outer circumferential member 29. That is, the outer circumferential member 29 functions as a frame for forming the backing material 28 during assembly of the ultrasound transmitting/receiving section 21.

The outer circumferential member 29 is made of a porous material having electric insulation. For example, the outer circumferential member 29 is desirably made of a material having high thermal conductivity such as alumina. As shown in FIG. 3 and FIG. 5, a part of the outer circumferential member 29 is exposed to an inside of the heat receiving chamber 25.

A part of the electric circuit board 31 is embedded in the backing material 28. The electric circuit board 31 is fixed to the ultrasound transmitting/receiving section 21 by the backing material 28. That is, a part of the electric circuit board 31 projects into the heat receiving chamber 25 from the backing material 28.

The electric circuit board 31 is a member configured to relay electric connection between each of the plurality of ultrasound transducers 22 and respective coaxial cables included in the coaxial cable bundle 23. As shown in FIG. 3, the ultrasound transducer 22 and the electric circuit board 31 are electrically connected by a conductive wire 32 embedded in the backing material 28.

Core wires 23a of the respective coaxial cables of the coaxial cable bundle 23 are electrically connected to the electric circuit board 31. Note that, although not shown in the figure, a wire for grounding is also electrically connected to the ultrasound transducer 22.

A connecting portion of the electric circuit board 31 and the coaxial cable bundle 23 is sealed by sealing resin 33 made of a material having electric insulation.

As explained above, a part of an outer surface of the ultrasound transmitting/receiving section 21, which is the heat generating section, is exposed in the heat receiving chamber 25, which is the hollow section, provided in the holding section 24. More specifically, in the present embodiment, at least a part of each of the outer circumferential member 29, the backing material 28, the electric circuit board 31, and the sealing resin 33 is exposed in the heat receiving chamber 25.

The endoscope 1 includes a heat radiation chamber 36, which is a hollow section, communicating with the heat receiving chamber 25. A part where the heat radiation chamber 36 is disposed is not particularly limited as long as the part is in the endoscope 1. However, in the present embodiment, as an example, the heat radiation chamber 36 is provided in the holding section 24 of the distal end portion 10.

As in the present embodiment, when the heat receiving chamber 25 and the heat radiation chamber 36 are provided in the holding section 24, the heat receiving chamber 25 and the heat radiation chamber 36 may have a form in which the heat receiving chamber 25 and the heat radiation chamber 36 are formed as continuous one hollow section or may have a form in which the heat receiving chamber 25 and the heat radiation chamber 36 are independent two hollow sections and connected by a relatively narrow passage.

In the present embodiment, as an example, as shown in FIG. 4, the heat radiation chamber 36 is provided further on the proximal end side than the heat receiving chamber 25. The heat radiation chamber 36 and the heat receiving chamber 25 communicate with each other through a passage 37 configured to pierce through the holding section 24.

The treatment instrument channel 17, which is a conduit, held by the holding section 24 is disposed to be adjacent to the heat radiation chamber 36 or pass through the heat radiation chamber 36. Note that the conduit adjacent to the heat radiation chamber 36 or passing through the heat radiation chamber 36 may be an air/water feeding channel for feeding fluid to an air/water feeding port. The conduit may be a conduit through which the optical fiber cable connected to the illuminating apparatus is inserted.

The heat radiation chamber 36 is sealed except a part communicating with the heat receiving chamber 25. That is, closed one space is formed by the heat receiving chamber 25 and the heat radiation chamber 36. A heat medium 38 that changes in phase from liquid to gas when exceeding a predetermined temperature is encapsulated in the space formed by the heat receiving chamber 25 and the heat radiation chamber 36.

A method of encapsulating the heat medium 38 in the space formed by the heat receiving chamber 25 and the heat radiation chamber 36 is not particularly limited. However, in the present embodiment, as an example, the heat medium 38 is introduced into the heat radiation chamber 36 through at least one hole section 45 communicating with the heat radiation chamber 36 from an outer surface of the holding section 24. The hole section 45 is sealed by, for example, a medium sealing member 46 formed by a male screw 46a, which screws in a female screw section formed in the hole section 45, and an adhesive 46b filled in the hole section 45. With such a configuration, since it is easy to remove the medium sealing member 46 from inside the hole section 45 and seal the hole section 45 again, it is possible to easily perform work for supplying and replacing the heat medium 38.

The heat medium 38 is fluid having inactivity and electric insulation and having a boiling point equal to or higher than 30° C. and equal to or lower than 50° c and is, for example, fluorine-based fluid such as fluorocarbon. More desirably, the boiling point of the heat medium 38 is equal to or higher than 30° C. and equal to or lower than 40° C. Since the heat medium 38 is inactive, the heat medium 38 does not deteriorate members configuring the ultrasound transmitting/receiving section 21 and the holding section 24 in contact with the heat medium 38.

In order to prevent or restrict a decrease of the heat medium 38, the holding section 24 is desirably made of a material through which the heat medium 38 less easily permeates or does not permeate such as metal, polyphenylsulfon resin, polysulfon resin, or polyether ether ketone resin. Note that, even when the holding section 24 is made of other materials, the decrease of the heat medium 38 can be prevented or restricted by forming coating of a metal thin film on inner wall surfaces of the heat receiving chamber 25 and the heat radiation chamber 36.

Note that, as in the present embodiment, when the treatment instrument channel 17, which is the conduit, passes through the heat radiation chamber 36, a part located in the heat radiation chamber 36 of the treatment instrument channel 17 is configured by a material through which the heat medium 38 less easily permeates or does not permeate or a metal thin film is coated on a surface of the part.

As shown in FIG. 3, a film 34 is provided on a surface exposed in the heat receiving chamber 25 of the backing material 28. The film 34 is a thin film made of, for example, aluminum, copper, silicon dioxide, or the like. The film 34 may have a form in which the film 34 is formed by vapor deposition or a form in which a sheet-like member is stuck to the film 34. Penetration of the heat medium 38 into the ultrasound transmitting/receiving section 21 is prevented by the film 34.

Since the heat medium 38 is encapsulated in the heat receiving chamber 25, the outer surface exposed in the heat receiving chamber 25 of the ultrasound transmitting/receiving section 21 is in contact with the heat medium 38.

When the ultrasound transducer 22 of the ultrasound transmitting/receiving section 21 is driven, the ultrasound transducer 22 generates heat. The heat generated by the ultrasound transducer 22 is transmitted to the outer surface exposed in the heat receiving chamber 25 of the ultrasound transmitting/receiving section 21.

Therefore, the ultrasound transmitting/receiving section 21 generates heat, whereby the heat medium 38 in contact with the ultrasound transmitting/receiving section 21 in the heat receiving chamber 25 is heated to boil. The heat medium 38 changes in phase from liquid to gas. By using heat of vaporization at the time when the heat medium 38 vaporizes on a contact surface with the ultrasound transmitting/receiving section 21, it is possible to efficiently transmit heat of the ultrasound transmitting/receiving section 21 to the heat medium 38.

The vaporized heat medium 38 enters the heat radiation chamber 36 provided on the proximal end side and is cooled in the heat radiation chamber 36 to be liquid again. In the heat radiation chamber 36, the heat medium 38 is cooled by heat radiation from the outer surface of the holding section 24 and heat transmission to the bending section 11. The heat medium 38 circulates between the heat receiving chamber 25 and the heat radiation chamber 36 according to a temperature difference between the heat receiving chamber 25 and the heat radiation chamber 36.

In this way, in the present embodiment, by using the heat medium 38 that changes in phase to gas at temperature during action of the ultrasound transmitting/receiving section 21, which is the heat generating section, it is possible to more efficiently perform the cooling of the ultrasound transmitting/receiving section 21, which is the heat generating section, provided at the distal end portion 10 of the insertion section 2 compared with a conventional technique for feeding, for example, a coolant, which is liquid.

If cooling efficiency of the ultrasound transmitting/receiving section 21 is improved, for example, an output of the ultrasound transmitting/receiving section 21 can be increased more than in the past. For example, when the heat generating section is a light source apparatus such as a light emitting diode, a light amount emitted by the light source apparatus can be increased more than in the past.

In the present embodiment, since it is unnecessary to provide, in the insertion section, a conduit for feeding a coolant for transferring heat as in the past, it is possible to further reduce a diameter of the insertion section 2 and make the insertion section 2 more flexible.

In the present embodiment, heat of the heat medium 38 is also transmitted to the treatment instrument channel 17, which is the conduit that passes through the heat radiation chamber 36. Therefore, if suction action of the endoscope 1 is executed, cooling efficiency of the heat medium 38 can be further improved. When a conduit for air feeding and/or water feeding passes through the heat radiation chamber 36, similarly, if action of air feeding and/or water feeding is executed, the cooling efficiency of the heat medium 38 can be further improved.

In the present embodiment, the outer circumferential member 29, which is a member in contact with the heat medium 38 of the ultrasound transmitting/receiving section 21, is configured by the porous material. Therefore, a surface of the outer circumferential member 29 has a rough surface structure on which fine unevenness is present. The heat medium 38 in a state in contact with the rough surface structure more easily boils than in a state in contact with a smooth surface. Therefore, the heat medium 38 more easily vaporizes. The cooling efficiency of the ultrasound transmitting/receiving section 21 can be improved.

Note that the rough surface structure may be formed by post-machining such as electric discharge machining and may be formed on surfaces of the film 34, the electric circuit board 31, and the sealing resin 33. If air pressure of the heat receiving chamber 25 and the heat radiation chamber 36 is set to a state lower than an atmospheric pressure, it is possible to lower the boiling point of the heat medium 38 and allow the heat medium 38 to more easily vaporize.

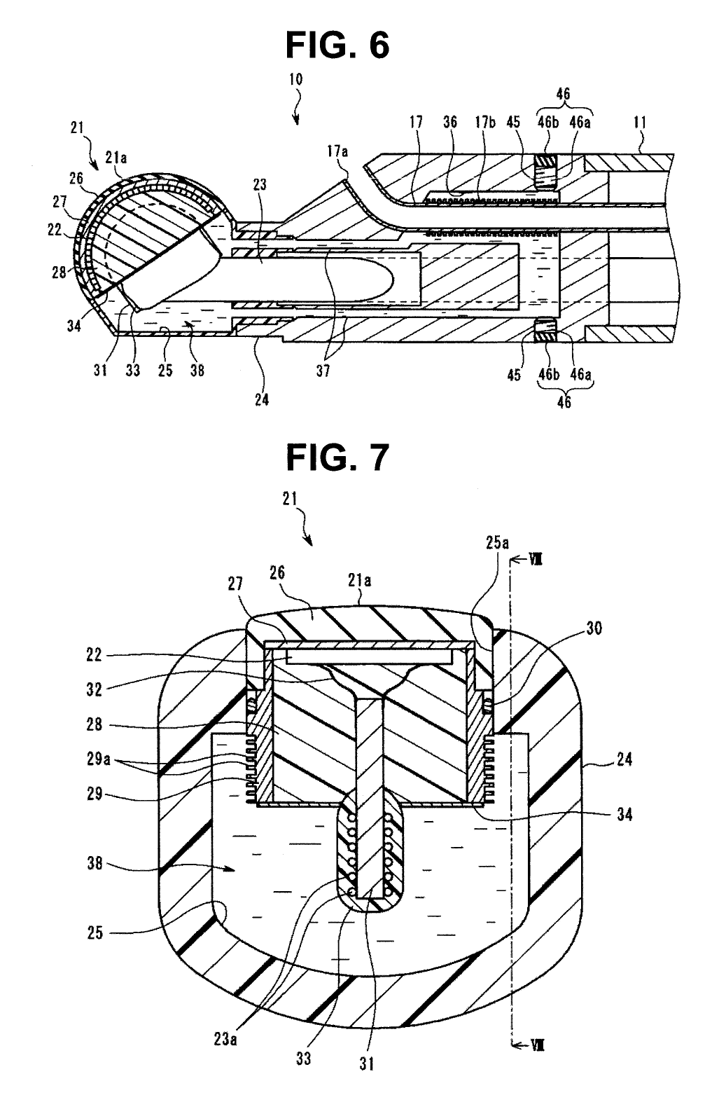

As shown in FIG. 6 as a modification, if an uneven section for increasing a contact area is provided in the member in contact with the heat medium 38 in the heat radiation chamber 36, the cooling of the heat medium 38 can be efficiently performed. In the modification shown in FIG. 6, an uneven section 17b, which is a plurality of fins, is provided on an outer circumferential surface of the treatment instrument channel 17 that passes through the heat radiation chamber 36.

The uneven section 17b is not limited to a fin-like section and may be, for example, a section made of a porous member. The uneven section may be provided on an inner wall surface of the heat radiation chamber 36.

### Second Embodiment

A second embodiment of the present invention is explained below. In the following explanation, only differences from the first embodiment are explained. Components same as the components in the first embodiment are denoted by the same reference numerals and signs and explanation of the components is omitted as appropriate.

The present embodiment is different from the first embodiment in that a plurality of slits are provided on a surface of a member in contact with the heat medium 38 of the ultrasound transmitting/receiving section 21.

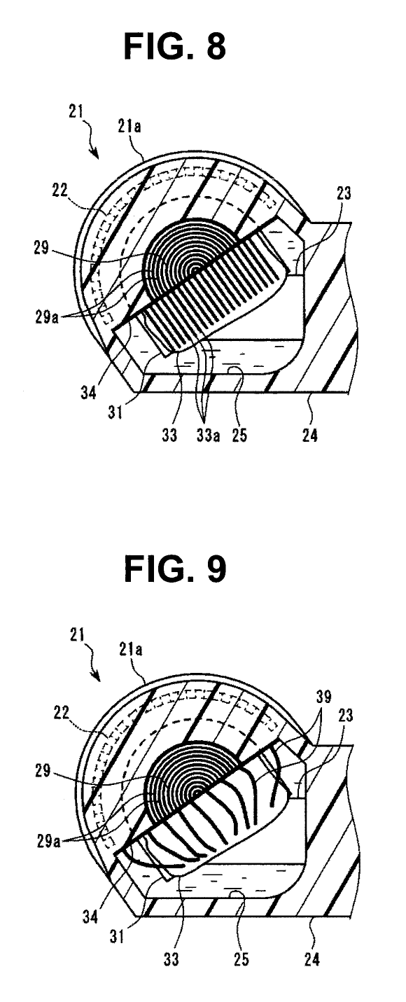

As shown in FIG. 7 and FIG. 8, in the present embodiment, a plurality of slits 29a are formed on a surface in contact with the heat medium 38 of the outer circumferential member 29. A plurality of slits 33a are formed on a surface of the sealing resin 33 as well. The slits 29a and 33a are set to width in which the heat medium 38, which is liquid, moves on insides according to capillarity.

In this way, the pluralities of slits 29a and 33a are provided on the surface of the ultrasound transmitting/receiving section 21. Consequently, it is possible to cause the heat medium 38, which is liquid, to reach, according to the capillarity, even a region located in gas present in the heat receiving chamber 25 on the surface of the ultrasound transmitting/receiving section 21.

Therefore, even in a state in which the gas is present in the heat receiving chamber 25, the heat medium 38, which is liquid, can be brought into contact with the ultrasound transmitting/receiving section 21, which is the heat generating section, irrespective of a posture of the distal end portion 10. The cooling of the ultrasound transmitting/receiving section 21 can be performed.

Since the pluralities of slits 29a and 33a are provided on the surface of the ultrasound transmitting/receiving section 21, an area of contact of the ultrasound transmitting/receiving section 21 and the heat medium 38 increases. Therefore, a heat quantity per unit time period transmitted from the ultrasound transmitting/receiving section 21 to the heat medium 38 can be increased. Cooling efficiency can be improved.

In the embodiment shown in the figure, as an example, the plurality of slits 29a are formed in parallel. However, the plurality of slits 29a may be formed to cross in a mesh shape. Similarly, the plurality of slits 33a may also be formed to cross in a mesh shape.

In the present embodiment as well, as shown in FIG. 6, the uneven section 17b, which is the plurality of fins, may be provided on the outer circumferential surface of the treatment instrument channel 17 that passes through the heat radiation chamber 36.

Note that, as in the first embodiment, the endoscope 1 in the present embodiment can more efficiently perform the cooling of the ultrasound transmitting/receiving section 21, which is the heat generating section, provided at the distal end portion 10 of the insertion section 2 compared with the conventional technique for feeding the coolant, which is liquid, for example.

### Third Embodiment

A third embodiment of the present invention is explained below. In the following explanation, only differences from the first and second embodiments are explained. Components same as the components in the first and second embodiments are denoted by the same reference numerals and signs and explanation of the components is omitted as appropriate.

As shown in FIG. 9, in the present embodiment, a plurality of fiber members 39 are disposed in a contact state on the surface of the ultrasound transmitting/receiving section 21 exposed in the heat receiving chamber 25.

In the present embodiment, as an example, the fiber members 39 are members inserted through the coaxial cable bundle 23 and configured to prevent extensional deformation of the coaxial cable bundle 23 at the time when tension is applied to the coaxial cable bundle 23.

In the present embodiment, since the heat medium 38, which is liquid, moves in the fiber members 39 according to capillarity, it is possible to cause the heat medium 38 to reach even a region located in gas present in the heat receiving chamber 25 on the surface of the ultrasound transmitting/receiving section 21.

Therefore, even in a state in which gas is present in the heat receiving chamber 25, the heat medium 38, which is liquid, can be brought into contact with the ultrasound transmitting/receiving section 21, which is the heat generating section, irrespective of a posture of the distal end portion 10. The cooling of the ultrasound transmitting/receiving section 21 can be performed.

In the present embodiment as well, as shown in FIG. 6, the uneven section 17b, which is the plurality of fins, may be provided on the outer circumferential surface of the treatment instrument channel 17 that passes through the heat radiation chamber 36. As in the second embodiment, in the present embodiment, a plurality of slits 33a may be provided on the surface of the sealing resin 33.

Note that, as in the first embodiment, the endoscope 1 in the present embodiment can more efficiently perform the cooling of the ultrasound transmitting/receiving section 21, which is the heat generating section, provided at the distal end portion 10 of the insertion section 2 compared with the conventional technique for feeding the coolant, which is liquid, for example.

### Fourth Embodiment

A fourth embodiment of the present invention is explained below. In the following explanation, only differences from the first, second, and third embodiments are explained below. Components same as the components in the first, second, and third embodiments are denoted by the same reference numerals and signs and explanation of the components is omitted as appropriate.

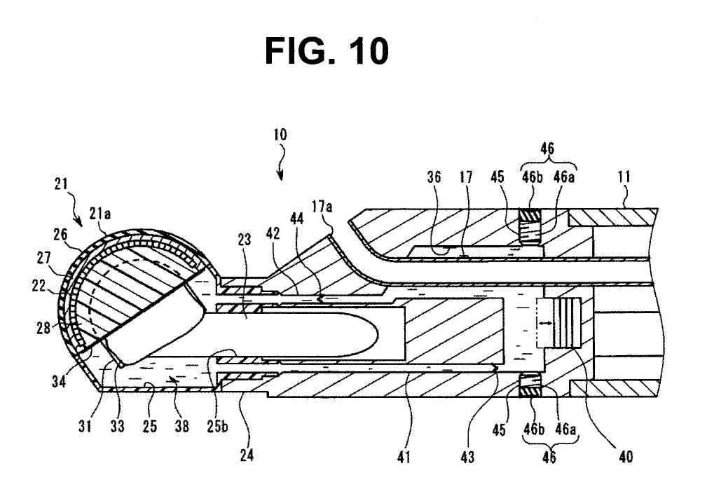

The endoscope 1 in the present embodiment shown in FIG. 10 is different from the endoscopes in the first, second, and third embodiments in that the endoscope 1 includes, in the distal end portion 10, an actuator 40 configured to move the heat medium 38 between the heat receiving chamber 25 and the heat radiation chamber 36.

In the present embodiment, the heat receiving chamber 25 and the heat radiation chamber 36 communicate with each other through a first conduit 41 and a second conduit 42, which are two independent conduits. In the first conduit 41, a first check valve 43 configured to allow a flow of fluid in the first conduit 41 only in a direction from the heat receiving chamber 25 toward the heat radiation chamber 36 is provided. In the second conduit 42, a second check valve 44 configured to allow a flow of fluid in the second conduit 42 only in a direction from the heat radiation chamber 36 toward the heat receiving chamber 25 is provided.

The actuator 40 changes a volume of the heat radiation chamber 36. The actuator 40 is, for example, a laminated piezoelectric device configured to expand and contract with energization. The actuator 40 causes a piston or a diaphragm provided on a wall surface of the heat radiation chamber 36 to project and recess in the heat radiation chamber 36. That is, the expansion and contraction of the actuator 40 is repeated, whereby the volume of the heat radiation chamber 36 changes in a pulsating manner.

If the volume of the heat radiation chamber 36 is changed in an increasing direction according to action of the actuator 40, the heat medium in the heat receiving chamber 25 flows into the heat radiation chamber 36 through the first conduit 41 according to a decrease in pressure in the heat radiation chamber 36. If the volume of the heat radiation chamber 36 is changed in a decreasing direction according to the action of the actuator 40, the heat medium 38 in the heat radiation chamber 36 flows into the heat receiving chamber 25 through the second conduit 42 according to an increase in the pressure in the heat radiation chamber 36. Therefore, in the present embodiment, the expansion and contraction of the actuator 40 is repeated, whereby the heat medium 38 circulates between the heat receiving chamber 25 and the heat radiation chamber 36.

That is, the endoscope 1 in the present embodiment includes, in the distal end portion 10, a configuration of a so-called volume pump. According to the present embodiment, a heat quantity per unit time period transferred by the heat medium 38 can be increased with respect to the first, second, and third embodiments.

In the present embodiment as well, as shown in FIG. 6, the uneven section 17b, which is the plurality fins, may be provided on the outer circumferential surface of the treatment instrument channel 17 that passes through the heat radiation chamber 36. As in the second embodiment, in the present embodiment, a plurality of slits may be provided on the surface of the ultrasound transmitting/receiving section 21 in the heat receiving chamber 25. As in the third embodiment, in the present embodiment, the fiber members 39 may be disposed on the surface of the ultrasound transmitting/receiving section 21 in the heat receiving chamber 25.

### Fifth Embodiment

A fifth embodiment of the present invention is explained below. In the following explanation, only differences from the fourth embodiment are explained. Components same as the components in the fourth embodiment are denoted by the same reference numerals and signs and explanation of the components is omitted as appropriate.

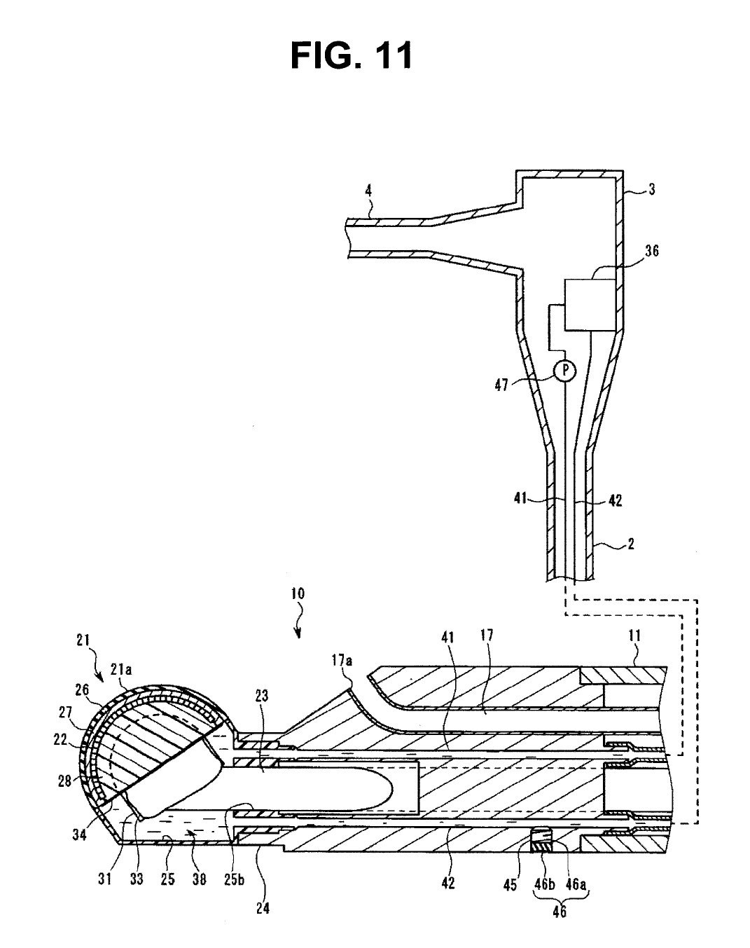

In the first to fourth embodiments explained above, the heat radiation chamber 36 is disposed at the distal end portion 10 of the insertion section 2. However, the heat radiation chamber 36 may be disposed in other parts in the endoscope 1. In the present embodiment, as an example, as shown in FIG. 11, the heat radiation chamber 36 is disposed in the operation section 3.

The heat receiving chamber 25 and the heat radiation chamber 36 communicate with each other through the first conduit 41 and the second conduit 42, which are the two independent conduits, inserted through the insertion section 2. In the operation section 3, a pump 47 is provided in the first conduit 41. The pump 47 operates to thereby transfer the heat medium 38 in the first conduit 41 from the heat receiving chamber 25 toward the heat radiation chamber 36. In the first conduit 41, since the heat medium 38 is in a state in which gas and liquid are mixed, the pump 47 desirably has a form of a gear pump or a piston pump adapted to transfer of fluid in a gas-liquid mixed state.

By operating the pump 47, the heat medium 38 circulates between the heat receiving chamber 25 and the heat radiation chamber 36. The cooling of the ultrasound transmitting/receiving section 21 can be performed. In the present embodiment, by disposing the heat radiation chamber 36 and the pump 47 in the operation section 3, the distal end portion 10 of the insertion section 2 can be reduced in size. In the operation section 3, the heat radiation chamber 36 and the pump 47 can be formed relatively large. Therefore, it is possible to improve efficiency of the cooling of the heat medium 38 and increase a flow rate of the heat medium 38. It is possible to cool the ultrasound transmitting/receiving section 21 to lower temperature.

Note that, as in the first to fourth embodiments explained above, in the present embodiment, a conduit may be inserted through the heat radiation chamber 36. The heat radiation chamber 36 and the pump 47 may be disposed in other parts in the endoscope 1, for example, in the endoscope connector 4a and in the ultrasound connector 6a.

In the present embodiment as well, as shown in FIG. 6, the uneven section 17b, which is the plurality of fins, may be provided on the outer circumferential surface of the treatment instrument channel 17 that passes through the heat radiation chamber 36. As in the second embodiment, in the present embodiment, a plurality of slits may be provided on the surface of the ultrasound transmitting/receiving section 21 in the heat receiving chamber 25. As in the third embodiment, in the present embodiment, the fiber members 39 may be disposed on the surface of the ultrasound transmitting/receiving section 21 in the heat receiving chamber 25.

The present invention is not limited to the embodiments explained above and can be changed as appropriate without departing from the gist or the idea of the invention read from the claims and the entire specification. Endoscopes involving such changes are also included in the technical scope of the present invention.

What is claimed is:

1. An endoscope comprising:
an insertion section configured to be inserted into a subject;
a distal end portion disposed at a distal end of the insertion section, the distal end portion including an observing section configured to observe the subject, a holding section configured to hold the observing section, a heat medium that changes in phase from liquid to gas when exceeding a predetermined temperature, and a heat receiving chamber configured to house the heat medium; and
a heat radiation chamber communicating with the heat receiving chamber, the heat medium gasified in the heat receiving chamber being capable of entering the heat radiation chamber.

2. The endoscope according to claim 1, wherein temperature at which the heat medium changes in phase from the liquid to the gas is equal to or higher than 30° C. and equal to or lower than 50° C.

3. The endoscope according to claim 2, further comprising a conduit configured to be inserted through the insertion section, wherein the conduit is disposed to be adjacent to the heat radiation chamber or pass through the heat radiation chamber.

4. The endoscope according to claim 2, wherein a surface of a member exposed in the heat receiving chamber has a rough surface structure at least in a part.

5. The endoscope according to claim 2, wherein a plurality of slits, in which the heat medium moves according to capillarity, are formed in at least a part of a surface of a member exposed in the heat receiving chamber.

6. The endoscope according to claim 2, wherein a fiber member is disposed in a contact state in at least a part of a surface of a member exposed in the heat receiving chamber.

7. The endoscope according to claim 2, further comprising an actuator configured to change a volume of the heat radiation chamber.

8. The endoscope according to claim 7, further comprising:
a first conduit configured to cause the heat receiving chamber and the heat radiation chamber to communicate with each other;
a first check valve provided in the first conduit and configured to allow a flow of fluid in the first conduit only in a direction from the heat receiving chamber toward the heat radiation chamber;
a second conduit provided independently from the first conduit and configured to cause the heat receiving chamber and the heat radiation chamber to communicate with each other; and
a second check valve provided in the second conduit and configured to allow a flow of fluid in the second conduit only in a direction from the heat radiation chamber toward the heat receiving chamber.

9. The endoscope according to claim 2, wherein the observing section includes at least an ultrasound transmitting/receiving section configured to transmit and receive ultrasound.

10. An endoscope comprising:
an ultrasound transmitting/receiving section; and
a holding section configured to form a heat receiving chamber, which is a space in which a heat medium that changes in phase from liquid to gas when exceeding a predetermined temperature is encapsulated, between the holding section and the ultrasound transmitting/receiving section and hold the ultrasound transmitting/receiving section in the heat medium such that at least a part of an outer surface of the ultrasound transmitting/receiving section is exposed to the heat receiving chamber, wherein
a plurality of slits are formed in a portion in contact with the heat medium in the ultrasound transmitting/receiving section.

11. The endoscope according to claim 10, wherein the plurality of slits cause the liquid heat medium to enter an outer circumferential member of the ultrasound transmitting/receiving section according to capillarity.
